(19) United States
(12) Patent Application Publication
(10) Pub. No.: US 2026/0348981 A1
(43) Pub. Date: Nov. 5, 2026

(54) DETECTION AND TREATMENT OF AATD-LD

(71) Applicant: Takeda Pharmaceutical Company Limited, Osaka (JP)

(72) Inventors: Virginia C. Clark, Gainesville, FL (US); May Hagiwara, Cambridge, MA (US); Karki Chitra, Winchester, MA (US); Rohit Loomba, San Diego, CA (US); Ed Gustavo Marins, Sudbury, MA (US); Suna Park, Cambridge, MA (US)

(73) Assignee: Takeda Pharmaceutical Company Limited, Osaka (JP)

(21) Appl. No.: 19/486,874

(22) PCT Filed: May 23, 2024
(86) PCT No.: PCT/IB2024/000274

§ 371 Date: Nov. 21, 2025

Related U.S. Application Data
(60) Provisional application No. 63/503,853, filed on May 23, 2023.
(60) Provisional application No. 63/589,168, filed on Oct. 10, 2023.

(51) Int. Cl. G01N 33/68; C12N 15/113; C12Q 1/6883

(52) U.S. Cl. CPC G01N 33/6893; C12N 15/113; C12Q 1/6883; C12N 2310/14; C12Q 2600/156; G01N 2333/8125; G01N 2800/085; G01N 2800/50

(57) ABSTRACT

The present disclosure provides methods for identifying individuals at risk for developing Alpha-1 Antitrypsin Deficiency-associated liver disease (AATD-LD), diagnosing individuals with AATD-LD, and administering therapeutic agents to individuals having or predisposed to AATD-LD.

### BACKGROUND

Alpha-1 Antitrypsin Deficiency-associated liver disease (AATD-LD) may develop in individuals having mutations in the SERPINA1 gene, which results in misfolded alpha-1 antitrypsin (AAT) protein. Misfolded AAT is poorly secreted from the liver, where it builds up and can cause liver injury and liver conditions such as hepatitis, cirrhosis, and an increased risk of liver cancer.

### SUMMARY

AAT deficiency (AATD) occurs with a frequency of about 1 in every 1,500 to 3,500 individuals and can result in the development of liver disease (AATD-LD) over the course of an individual's life. Diagnosis of AATD-LD is challenging due to asymptomatic progression of the disease, and predicting AATD-affected individuals who will develop liver disease is difficult due to variable risk among the population. Additionally, AATD often presents with symptoms that resemble other disease states, such as chronic obstructive pulmonary disease (COPD) or cryptogenic liver disease (cirrhosis of an unknown origin). Nevertheless, early detection and diagnosis of AATD-LD is critical, as late detection can lead to significant liver damage and complications, such as cirrhosis, liver cancer, or liver failure. Thus, there exists a need to develop robust diagnostic tools that can identify individuals with AATD-LD early in disease progression and/or predict which individuals with AATD may develop liver disease.

The present disclosure provides methods for determining the likelihood of an individual to develop AATD-LD, diagnosing AATD-LD, and administering treatment for AATD-LD. In some aspects, provided herein are methods for identifying an individual that has or is predisposed to develop Alpha-1 Antitrypsin Deficiency Associated Liver Disease (AATD-LD), the method comprising determining at least two of the following indicators:
a. Previous diagnosis of AATD,
b. Blood AST levels above about 34 U/L,
c. Weight above about 180 pounds or below about 100 pounds,
d. Blood alanine transaminase (ALT) levels above about 30 U/L,
e. Body Mass Index (BM) below about 18.5 or above about 25,
f. Racial identity of Caucasian,
g. Serum alpha-1 antitrypsin (AAT) levels below about 100 mg/dL,
h. Mean blood sodium levels less than about 135 mEq/L or greater than about 145 mEq/L,
i. Age of over about 40 years, or
j. Proton pump inhibitor (PPI) use within about 100 days of assessment.
   The presence of a plurality of indicators increases the likelihood that an individual has or will develop AATD-LD.

In some aspects, provided herein are methods for identifying an individual that has or is predisposed to develop Alpha-1 Antitrypsin Deficiency Associated Liver Disease (AATD-LD), the method comprising determining at least three of the following indicators:
a. Previous diagnosis of AATD,
b. Blood AST levels above about 34 U/L,
c. Weight above about 180 pounds or below about 100 pounds,
d. Blood alanine transaminase (ALT) levels above about 30 U/L,
e. Body Mass Index (BMI) below about 18.5 or above about 25,
f. Racial identity of Caucasian,
g. Serum alpha-1 antitrypsin (AAT) levels below about 100 mg/dL,
h. Mean blood sodium levels less than about 135 mEq/L or greater than about 145 mEq/L,
i. Age of over about 40 years, or
j. Proton pump inhibitor (PPI) use within about 100 days of assessment.

In some aspects, provided herein are methods for identifying an individual that has or is predisposed to develop Alpha-1 Antitrypsin Deficiency Associated Liver Disease (AATD-LD), the method comprising determining existence of previous AATD diagnosis and the presence of blood aspartate aminotransferase (AST) levels above about 34 U/L.

In some aspects, provided herein are methods for identifying an individual that has or is predisposed to develop AATD-LD, the method comprising determining existence of previous AATD diagnosis, the presence of blood AST levels above about 34 U/L, and body weight above about 180 pounds or below about 100 pounds.

In some aspects, provided here are methods for identifying an individual that has or is predisposed to develop AATD-LD, the method comprising determining existence of previous AATD diagnosis and the presence of at least one of:
a. Blood AST levels above about 34 U/L,
b. Weight above about 180 pounds or below about 100 pounds,
c. Blood alanine transaminase (ALT) levels above about 30 U/L,
d. Body Mass Index (BMI) below about 18.5 or above about 25,
e. Racial identity of Caucasian,
f. Serum alpha-1 antitrypsin (AAT) levels below about 100 mg/dL,
g. Mean blood sodium levels less than 135 mEq/L or greater than about 145 mEq/L,
h. Age of over about 40 years, or
i. Proton pump inhibitor (PPI) use within about 100 days of assessment.

In some aspects, provided herein are methods for identifying an individual that has or is predisposed to develop AATD-LD, the method comprising determining existence of previous AATD diagnosis and the presence of at least two of:
a. Blood AST levels above about 34 U/L,
b. Weight above about 180 pounds or below about 100 pounds,
c. Blood ALT levels above about 30 U/L,
d. BMI below about 18.5 or about above 25,
e. Racial identity of Caucasian,
f. Serum AAT levels below about 100 mg/dL,
g. Mean blood sodium levels less than about 135 mEq/L or greater than about 145 mEq/L,
h. Age of over about 40 years, or
i. PPI use within about 100 days of assessment.

In some embodiments, the individual has blood AST levels above about 34 U/L. In some embodiments, the individual has blood AST levels above about 34 U/L and weight above about 180 pounds. In some embodiments, the individual has blood AST levels above about 34 U/L and weight below about 100 pounds.

In some aspects, provided herein are methods for identifying an individual that has or is predisposed to develop AATD-LD, the method comprising determining existence of previous AATD diagnosis and the presence of:
a. Blood AST levels above about 34 U/L,
b. Weight above about 180 pounds or below about 100 pounds,
c. Blood ALT levels above about 30 U/L,
d. BMI below about 18.5 or above about 25,
e. Racial identity of Caucasian,
f. Serum AAT levels below about 100 mg/dL,
g. Mean blood sodium levels less than about 135 mEq/L or greater than about 145 mEq/L,
h. Age of over about 40 years, and
i. PPI use within about 100 days of assessment.

In some aspects, provided herein are methods for determining the likelihood of developing liver disease for a patient with AATD, the method comprising determining the presence of at least one of:
a. Blood AST levels above about 34 U/L,
b. Weight above about 180 pounds or below about 100 pounds,
c. Blood ALT levels above about 30 U/L,
d. BMI below about 18.5 or above about 25,
e. Racial identity of Caucasian,
f. Serum AAT levels below about 100 mg/dL,
g. Mean blood sodium levels less than about 135 mEq/L or greater than about 145 mEq/L,
h. Age of over about 40 years, or
i. PPI use within about 100 days of assessment.

In some embodiments, the methods for identifying an individual that has or is predisposed to develop AATD-LD comprise ex vivo measurements. For example, methods of determining blood or serum levels of AST, ALT, and sodium levels are performed ex vivo on blood samples from individuals. In some embodiments, the method further comprises genotyping the patient or the individual for a SERPINA1 gene mutation. In some embodiments, the gene mutation is a PiZ mutation.

In some aspects, provided herein are methods of treating, preventing, or inhibiting the progression of AATD-LD in a subject having AATD and blood AST levels above about 34 U/L, the methods comprising administering to the subject an agent that reduces AAT levels.

In other aspects, provided herein are methods of treating or preventing AATD-LD in a subject having AATD and weight above about 180 pounds or below about 100 pounds, the methods comprising administering to the subject an agent that reduces AAT levels.

In some embodiments, the agent is an siRNA. In some embodiments, the agent is TAK-999 (Fazisiran).

In some embodiments, provided herein is an agent that reduces AAT levels for use in treating a subject identified to have or be predisposed to AAT-LD. In some embodiments provided herein is TAK-999 (Fazisiran) for use in treating a subject identified to have or be predisposed to develop AATD-LD. In some embodiments, provided herein is an agent (e.g., TAK-999) for use in a method of treating an individual that has or is predisposed to develop AAT-LD, wherein the individual has been identified to have AATD, blood AST levels above about 34 U/L, weight above about 180 pounds, or any combinations thereof. In some embodiments, provided herein is an agent (e.g., TAK-999) for use in a method of treating an individual that has or is predisposed to develop AAT-LD, wherein the individual has been identified to have AATD, blood AST levels above about 34 U/L, weight below about 100 pounds, or any combinations thereof.

### BRIEF DESCRIPTION OF THE DRAWINGS

FIG. 1 depicts the chemical structure of NAG37 (N-acetyl-galactosamine 37).

FIG. 2 shows the top 10 features associated with increased risk of undiagnosed AATD-LD and their proportion of total feature importance.

FIG. 3 illustrates the relative risk of developing AATD-LD as a function of the top three most-predictive model features: AATD diagnosis encounters, most recent AST blood levels, and body weight.

FIG. 4 shows the area under the receiver operator characteristic curve (area under the curve (AUC)=0.82).

### DETAILED DESCRIPTION

Unless otherwise defined herein, scientific and technical terms used herein have the meanings that are commonly understood by those of ordinary skill in the art. In the event of any latent ambiguity, definitions provided herein take precedence over any dictionary or extrinsic definition. Unless otherwise required by context, singular terms shall include pluralities and plural terms shall include the singular. The use of “or” means “and/or” unless stated otherwise. The use of the term “including”, as well as other forms of the term, is not limiting.

### DEFINITIONS

In order that the present disclosure may be more readily understood, certain terms are first defined. Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition.

As used herein, the singular forms “a”, “an” and “the” include plural referents unless the context clearly dictates otherwise. “And” as used herein is interchangeably used with “or” unless expressly stated otherwise. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately,” as applied to one or more values provided herein, refers to a value that is similar to a stated reference value. In some embodiments, the term “about” or “approximately” refers to a range of values that fall within and include 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context. When “about” or “approximately” is present before a value, it is understood to also include that reference value. In some embodiments, “about” or “approximately” can be understood as about 2 standard deviations from the mean. In some embodiments, “about” or “approximately” means up to and including ±10% (e.g., ±10%, ±9%, ±8%, ±7%, ±6%, ±5%, ±4%, ±3%, ±2%, ±1%, or less). In some embodiments, “about” or “approximately” means ±5%. When “about” or “approximately” is present before a series of numbers or a range, it is understood that it can modify each of the numbers in the series or range.

The term “administering” or “administration of,” as used herein, means to provide an agent, e.g., RNA interference (RNAi) agent or conjugate to a subject. In some embodiments, “administering” or “administration of” means to provide an RNAi agent or conjugate to a subject in a manner that is physiologically and/or (e.g., and) pharmacologically useful (e.g., to treat a condition in the subject). Non-limiting examples of routes of administration include intravenous, intramuscular, intraperitoneal, intracerebrospinal, subcutaneous, intra-articular, intrasynovial, or intrathecal routes. In some embodiments, the route of administration is subcutaneous.

The term “antisense strand” or “guide strand” refers to a single stranded nucleic acid molecule, which is one strand of a double stranded RNAi molecule, and which comprises a region of complementarity to a target sequence (e.g., a target gene sequence. RNA sequence, or mRNA sequence). The antisense strand may contain modified nucleosides with base analogs and is not necessarily 100% complementary over its entire length to the target sequence, but must at least be sufficiently complementary to hybridize with a target RNA and result in RNA interference.

The terms “silence,” “reduce,” “inhibit,” “down-regulate,” or “knockdown” when referring to expression of a given gene (e.g., SERPINA1), mean that the expression of the gene, as measured by the level of RNA transcribed from the gene or the level of polypeptidic, protein or protein subunit translated from the mRNA in a cell, group of cells, tissue, organ, or subject in which the gene is transcribed, is reduced when the cell, group of cells, tissue, organ, or subject is treated with an RNAi agent or conjugate described herein as compared to a control or reference cell, group of cells, tissue, organ or a subject, e.g., a second cell, group of cells, tissue, organ, or subject that has not or have not been so treated. In some embodiments, when a cell, group of cells, tissue, organ, or subject is treated with an RNAi agent or conjugate described herein, expression of a target gene (e.g., SERPINA1) is reduced by at least 10%, at least 20%, at least 30%, at least 40%, at least 50%, at least 60%, at least 70%, at least 80%, at least 90% or at least 95% relative to a control, e.g. baseline level of gene expression prior to treatment.

The term “subject,” as used herein, refers to a mammal. In some embodiments, a subject is a human. In some embodiments, a subject is a patient, e.g., a human patient that has or is suspected of having a disease. In some embodiments, the subject is a human patient who has or is suspected of having an AATD-associated disease.

### Alpha-1 Antitrypsin Deficiency

Alpha-1 antitrypsin (AAT, al-antitrypsin, or A1AT) deficiency is an inherited, autosomal codominant genetic disorder that causes misfolding of the AAT protein and poor secretion of the misfolded protein leading to lung and liver diseases. AAT deficiency (AATD) occurs with a frequency of about 1 in every 1,500 to 3,500 individuals and most often affects persons with European ancestry. An estimated approximately 90% of individuals with AATD go undiagnosed in the United States alone, due to a number of obstacles (Ye and D'Urzo. Challenge of α₁-antitrypsin deficiency diagnosis in primary care, 2016, Can Fam Physician. v.62(11)). Symptoms are often consistent with chronic obstructive pulmonary disease (COPD) and cryptogenic liver disease (cirrhosis of uncertain etiology), and are therefore often not attributed to AATD, which is a comparatively rarer disease (Brantley et al. Detection of alpha-1 antitrypsin deficiency: the past, present and future. Orphanet J Rare Dis, 2020. 15(1):96). Symptoms in AATD patients are often highly variable, and AAT serum levels—typically the first initial screening test—is insufficient to identify patients who will develop severe lung and/or liver disease (Torres-Duran et al. Alpha-1 antitrypsin deficiency: outstanding questions and future directions. Orphanet J Rare Dis, 2018. 13(1):114). AAT serum levels increase with progressive COPD stages, further complicating discrimination between COPD and AATD. Additionally, there is no universally accepted laboratory algorithm for AATD diagnosis, and genotyping is limited in its detection of rare allelic mutations (Ye and D'Urzo, 2016). All of these factors often combine to result in a diagnostic delay of several years, during which time AATD can cause lung and/or liver disease of increasing severity.

AAT is a protease inhibitor belonging to the serpin superfamily. Normal AAT protein is a circulating glycoprotein protease inhibitor primarily synthesized in the liver by hepatocytes and secreted into the blood. The known physiologic function of AAT is to inhibit neutrophil proteases, which serves to protect host tissues from non-specific injury during periods of inflammation. AAT is encoded by the SERPINA1 gene, of which numerous genetic variations have been identified and may be found at, for example, NCBI dbSNP and UniProt (see, e.g., ncbi.nlm.nih.gov/snp). Worldwide, more than 190 million people are estimated to carry at least one of the SERPINA1 alleles, S or Z, associated with AATD, and at about 5.5 million people carrying two of these alleles (Pi*SS, Pi*SZ, or Pi*ZZ). Patients with AATD and a Pi*ZZ genotype are associated with the greatest risk of developing AATD liver disease (AATD-LD).

The most clinically significant form of AATD, which is associated with liver disease in children and adults, and pulmonary disease in adults, is caused by the Z mutation in the SERPINA1 gene. The Z mutant allele (PiZ), through a single point mutation, renders the mutant Z form AAT protein (the “Z-AAT protein”) prone to abnormal folding causing intracellular retention. The mutant Z-AAT protein monomers are able to form chains of polymers that a mass into aggregates, which are sometimes referred to as “globules.” The misfolded Z-AAT protein is ineffective in traversing the secretory pathway, and instead polymerizes and accumulates in the endoplasmic reticulum (ER) of hepatocytes. The polymeric globule masses stress the ER and trigger continuous hepatocyte injury, leading to fibrosis, cirrhosis, and increased risk of hepatocellular carcinoma. Further, the absence of circulating anti-protease activity leaves the lung vulnerable to injury by neutrophil elastase, resulting in the development of respiratory complications such as emphysema.

Individuals with the homozygous Pi*ZZ genotype have severe deficiency of functional AAT, which leads to pulmonary disease. Weekly use of AAT augmentation therapy, using purified human AAT, results in near normal plasma levels of AAT in subjects with AATD, and helps prevent lung damage in affected individuals. However, while the administration of purified AAT can ameliorate or help prevent lung damage caused by the absence of endogenously secreted AAT, AATD patients remain vulnerable to endoplasmic reticulum liver storage disease caused by the deposition and accumulation of excessive abnormally folded AAT protein. Accumulated Z-AAT protein in the globule conformation in hepatocytes is a well-known characteristic of AATD liver disease and is believed to lead to proteotoxic effects that are responsible for inducing liver injury, including liver cell damage and death and chronic liver injury, in individuals with AATD. (see, e.g., D. Lindblad et al., Hepatology 2007, 46: 1228-1235). Patients with AATD often develop liver disease, which can be severe or fatal, even in infancy. Clinical presentations of injury in the liver include chronic hepatitis, cirrhosis, hepatocellular carcinoma, transaminitis, cholestasis, fibrosis, and even fulminant hepatic failure. However, some individuals do not clinically present with symptoms of AATD-associated liver disease (AATD-LD), making diagnosis challenging, and individuals may experience lengthy delays and multiple physician visits before receiving a definitive diagnosis. Additionally, the risk of developing AATD-LD varies across individuals.

Accordingly, provided herein are indicators that predict individuals who may have undiagnosed AATD-LD. Such indicators may be used in the development of standardized testing, which allows earlier diagnosis and more effective treatments. The indicators were identified using an artificial intelligence/machine learning-informed clinical decision support tool (AI/ML-CDST), which was developed using the US IQVIA Ambulatory Electronic Medical Records (AEMR) database. In the initial development stage, 3843 clinical features were analyzed and later reduced to 400 features in a subsequent iteration of the model. The features included were:
Patient characteristics: demographic and geographic information (11 features);
Knowledge-driven: predictors generated based on existing literature/clinical knowledge/hypotheses and aggregated to pre-defined groups of clinical codes (46 features);
Data-driven: predictors generated without any pre-existing clinical knowledge and aggregated to existing levels of clinical hierarchy (303 features); and
Custom: predictors that are not knowledge- or data-driven (40 features).

The feature profiles of the positive and negative cohorts were compared using descriptive statistics. Other elements of the model development are described in the Examples.

Among the factors that are evaluated when considering AATD-LD are levels of sodium, aspartate aminotransferase (AST), and/or alanine transaminase (ALT) in the blood and levels of AAT in the serum. In some aspects, the present disclosure provides methods for identifying an individual that has or is predisposed to develop Alpha-1 Antitrypsin Deficiency Associated Liver Disease (AATD-LD), the method comprising determining existence of previous AATD diagnosis and the presence of blood aspartate aminotransferase (AST) levels above about 34 U/L. In other aspects, the present disclosure provides methods of treating or preventing AATD-LD in a subject having AATD and blood AST levels above about 34 U/L, the method comprising administering to the subject an agent that reduces AAT levels.

Further indicators that may be considered in assessing for AATD-LD may be body mass index (BMI), body weight, an individual's use of proton pump inhibitors (PPI), and an individual's race and/or age. The factors identified in the present disclosure as being predictors of identifying an individual having or being predisposed to AATD-LD are listed in Table 1.

TABLE 1
Ten predictors for an individual having or being predisposed to AATD-LD.

| | Threshold at which individual has an |
|---|---|
| | increased likelihood of having or developing |
| Predictor | AATD-LD |
| AATD diagnosis | At least one instance of AATD diagnosis |
| AST | Above about 34 U/L |
| Weight | Greater than about 180 pounds or less than |
| | about 100 pounds |
| ALT | Above about 30 U/L |
| BMI | Below about 18.5 or above about 25 |
| Race (Caucasian) | Individual reporting as Caucasian |
| Individual's Age | Greater than about 40 |
| Serum AAT | Below about 100 mg/dL |
| Sodium | Mean value below about 135 mEq/L or above |
| | about 145 mEq/L |
| PPI | Use of PPIs within about 100 days prior to |
| | assessment |

In some aspects, the present disclosure provides methods for identifying an individual that has or is predisposed to develop AATD-LD, the method comprising determining existence of previous AATD diagnosis and the presence of:
a. Blood AST levels above about 34 U/L,
b. Weight above about 180 pounds or below about 100 pounds,
c. Blood ALT levels above about 30 U/L,
d. BMI below about 18.5 or above about 25,
e. Racial identity of Caucasian,
f. Serum AAT levels below about 100 mg/dL,
g. Mean blood sodium levels less than about 135 mEq/L or greater than about 145 mEq/L,
h. Age of over about 40 years, and/or
i. PPI use within about 100 days of assessment.

Therapeutic modalities for AATD-LD are limited and non-specific, and those currently in use include supportive care to ameliorate liver damage (such as changes to diet); administration of corticosteroids to reduce inflammation; and, in severe instances of AATD-LD, liver transplantation. More specific treatments in development for AATD-LD include administering silencing RNAs (siRNAs) or other RNA interference (RNAi) therapies to reduce levels of AAT. SiRNA treatments include, but are not limited to Belcesiran, ALN-AAT02, and TAK-999 (also known as Fazisiran). The chemical duplex structure of the siRNA agent TAK-999 is shown below (see also U.S. Pat. No. 10,450,565 and International Patent Publication WO 2018/132432, each of which is incorporated by reference herein for additional description of AD04837 (TAK-999)):

### Sense Strand:

(NAG37)s(invAb)sagcguuuaGfGfCfauguuuaacas(invAb)

### Antisense Strand

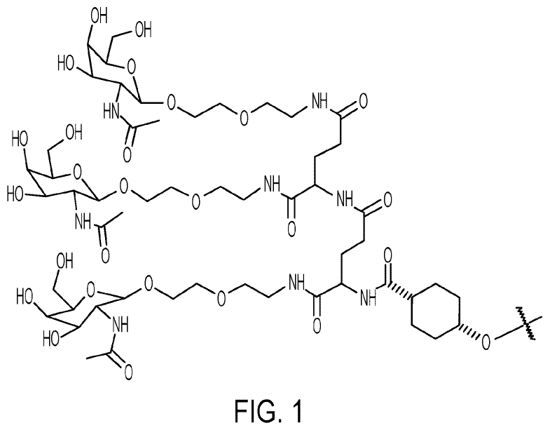

usGfsuUfaAfacaugCfcUfaAfaCfgCfsu
   Chemical modification legend: lowercase (n)=2′-O-methyl modified nucleotide; Nf=2′-Fluoro modified nucleotide; invAb=inverted abasic; s=phophorothioate.
   NAG37 has the structure as shown in FIG. 1.

In some aspects, the present disclosure provides a method of treating or preventing AATD-LD in a subject having AATD and blood AST levels above about 34 U/L, the method comprising administering to the subject an agent that reduces AAT levels. In other aspects, the present disclosure provides a method of treating or preventing AATD-LD in a subject having AATD and weight above about 180 pounds or below about 100 pounds, the method comprising administering to the subject an agent that reduces AAT levels. In still other aspects, the present disclosure provides a method of treating or preventing AATD-LD in a subject having AATD, blood AST levels above about 34 U/L, and weight above about 180 pounds or below about 100 pounds, the method comprising administering to the subject an agent that reduces AAT levels. In some embodiments, the agent is an siRNA. In some embodiments, the agent is TAK-999 (Fazisiran).

### EXAMPLES

### Example 1. Ten Predictors for AATD-LD Diagnosis

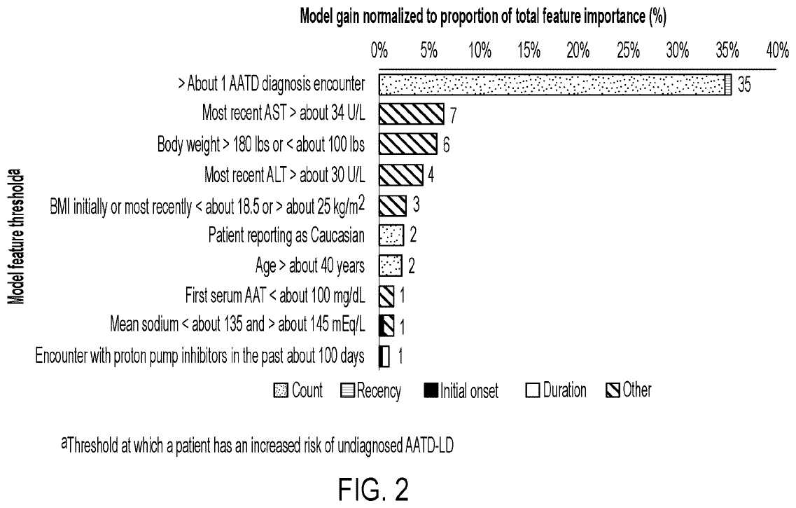

Diagnosis of alpha-1 antitrypsin deficiency (AATD)-associated liver disease (LD) is challenging due to the variable risk and asymptomatic nature of the disease. An artificial intelligence/machine learning informed clinical decision support tool (AI/ML-CDST) may inform the identification of patients at risk of having undiagnosed AATD-LD, even those whose liver enzymes (a primary biomarker of AATD-LD) are only mildly elevated. The first-stage development of the AI/ML-CDST using data from the US IQVIA Ambulatory Electronic Medical Records (AEMR) database is presented. Of the 400 features included, the top ten predictors of undiagnosed AATD-LD (FIG. 2) identified are also presented.

The study population comprised patients aged ≥18 years with ≥1 encounter for any medical activity in the IQVIA AEMR database from October 2015 to October 2022. Use of the IQVIA AEMR database to develop the AI/ML-CDST provided a larger and more diverse sample of patients compared to individual healthcare organizations, and the data therein are representative of the deployment data environment. The positive cohort comprised patients with a recorded AATD diagnosis (ICD-9/10 or SNOMED) and either a liver transplant or AATD-related liver conditions. The negative cohort comprised all other patients. For each patient cross-sectional data with 12-month lookback and outcome periods with 1-month shift were generated. A cross-section that included the first date of AATD-LD (based on liver transplant or AATD-related liver conditions) in the outcome period was considered a positive cohort cross-section. All other cross-sections were considered negative. Algorithm features were captured within the database and metrics were engineered for each variable. Data were split into training (preliminary modeling and parameter optimization), test, and hold-out sets, which help generate an unbiased measure of model performance of the final model on unseen data. A non-parametric XGBoost algorithm was developed using temporal cross-validation on the training and test set. Recursive feature elimination was performed to evaluate the impact of algorithm simplification on model performance. Final model performance was evaluated on the hold-out set and assessed by fold-improvement over incidence of AATD-LD observed in the IQVIA AEMR data.

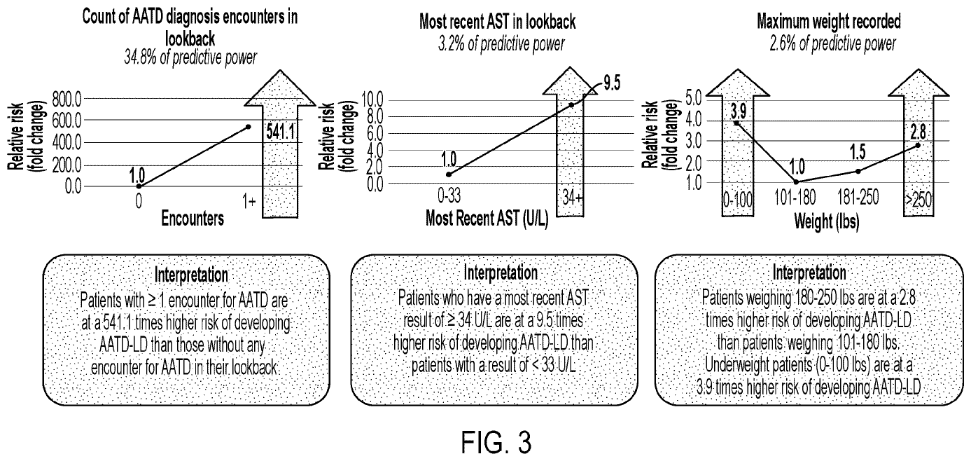

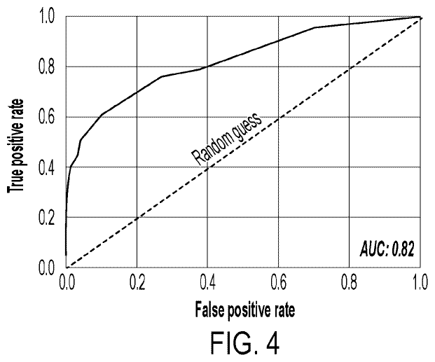

Utilizing the IQVIA AEMR database of 43,419,885 individuals, including 1,653 patients with AATD-LD, among the 400 features included to predict increased risk of underdiagnosed AATD-LD, prior AATD diagnosis was the most predictive (35%), followed by aspartate aminotransferase (AST) levels >about 34 U/L (7%) and body weight >about 180 or <about 100 pounds (6%) (Table 1) (FIGS. 2-3). Removal of AATD-LD encounters from the algorithm resulted in a significant impact on the algorithm sensitivity. The AI/ML-CDST achieved an area under the curve of 0.82 (FIG. 4), indicative of excellent performance in the identification of patients with AATD-LD. Other performance metrics for the AI/ML-CDST were specificity (99.99%), sensitivity (4.9%), positive predictive value (2.81%), negative predictive value (99.99%), and overall classification rate (99.99%).

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Where a range of values is provided, each value between and including the upper and lower ends of the range are specifically contemplated and described herein.

What is claimed is:

1. A method for identifying an individual that has or is predisposed to develop Alpha-1 Antitrypsin Deficiency Associated Liver Disease (AATD-LD), the method comprising determining at least two of the following indicators:
a. Previous diagnosis of AATD,
b. Blood AST levels above 34 about U/L,
c. Weight above about 180 pounds or below 100 pounds,
d. Blood alanine transaminase (ALT) levels above about 30 U/L,
e. Body Mass Index (BMI) below about 18.5 or above about 25,
f. Racial identity of Caucasian,
g. Serum alpha-1 antitrypsin (AAT) levels below about 100 mg/dL,
h. Mean blood sodium levels less than about 135 mEq/L or greater than about 145 mEq/L,
i. Age of over about 40 years, or
j. Proton pump inhibitor (PPI) use within about 100 days of assessment.

2. A method for identifying an individual that has or is predisposed to develop Alpha-1 Antitrypsin Deficiency Associated Liver Disease (AATD-LD), the method comprising determining at least three of the following indicators:
a. Previous diagnosis of AATD,
b. Blood AST levels above about 34 U/L,
c. Weight above about 180 pounds or below about 100 pounds,
d. Blood alanine transaminase (ALT) levels above about 30 U/L,
e. Body Mass Index (BMI) below about 18.5 or above about 25,
f. Racial identity of Caucasian,
g. Serum alpha-1 antitrypsin (AAT) levels below about 100 mg/dL,
h. Mean blood sodium levels less than about 135 mEq/L or greater than about 145 mEq/L,
i. Age of over about 40 years, or
j. Proton pump inhibitor (PPI) use within about 100 days of assessment.

3. A method for identifying an individual that has or is predisposed to develop Alpha-1 Antitrypsin Deficiency Associated Liver Disease (AATD-LD), the method comprising determining existence of previous AATD diagnosis and the presence of blood aspartate aminotransferase (AST) levels above about 34 U/L.

4. A method for identifying an individual that has or is predisposed to develop AATD-LD, the method comprising determining existence of previous AATD diagnosis, the presence of blood AST levels above about 34 U/L, and body weight above about 180 pounds or below about 100 pounds.

5. A method for identifying an individual that has or is predisposed to develop AATD-LD, the method comprising determining existence of previous AATD diagnosis and the presence of at least one of:
a. Blood AST levels above about 34 U/L,
b. Weight above about 180 pounds or below about 100 pounds,
c. Blood alanine transaminase (ALT) levels above about 30 U/L,
d. Body Mass Index (BMI) below about 18.5 or above about 25,
c. Racial identity of Caucasian,
f. Serum alpha-1 antitrypsin (AAT) levels below about 100 mg/dL,
g. Mean blood sodium levels less than about 135 mEq/L or greater than about 145 mEq/L,
h. Age of over about 40 years, or
i. Proton pump inhibitor (PPI) use within about 100 days of assessment.

6. A method for identifying an individual that has or is predisposed to develop AATD-LD, the method comprising determining existence of previous AATD diagnosis and the presence of at least two of:
a. Blood AST levels above about 34 U/L,
b. Weight above about 180 pounds or below about 100 pounds,
c. Blood ALT levels above about 30 U/L,
d. BMI below about 18.5 or above about 25,
e. Racial identity of Caucasian,
f. Serum AAT levels below about 100 mg/dL,
g. Mean blood sodium levels less than about 135 mEq/L or greater than about 145 mEq/L,
h. Age of over about 40 years, or
i. PPI use within about 100 days of assessment.

7. The method of any one of claims 1-2 and 5-6, wherein the individual has blood AST levels above about 34 U/L.

8. The method of any one of claims 1-3 and 5-6, wherein the individual has blood AST levels above about 34 U/L and weight above 180 pounds.

9. The method of any one of claims 1-3 and 5-7, wherein the individual has blood AST levels above 34 U/L and weight below about 100 pounds.

10. A method for identifying an individual that has or is predisposed to develop AATD-LD, the method comprising determining existence of previous AATD diagnosis and the presence of:
a. Blood AST levels above about 34 U/L,
b. Weight above about 180 pounds or below about 100 pounds,
c. Blood ALT levels above about 30 U/L,
d. BMI below about 18.5 or above about 25,
e. Racial identity of Caucasian,
f. Serum AAT levels below about 100 mg/dL,
g. Mean blood sodium levels less than about 135 mEq/L or greater than about 145 mEq/L,
h. Age of over about 40 years, and
i. PPI use within about 100 days of assessment.

11. A method for determining the likelihood of developing liver disease for a patient with AATD, the method comprising determining the presence of at least one of:
a. Blood AST levels above about 34 U/L,
b. Weight above about 180 pounds or below above 100 pounds,
c. Blood ALT levels above about 30 U/L,
d. BMI below about 18.5 or above about 25,
e. Racial identity of Caucasian,
f. Serum AAT levels below about 100 mg/dL,
g. Mean blood sodium levels less than about 135 mEq/L or greater than about 145 mEq/L,
h. Age of over about 40 years, or
i. PPT use within about 100 days of assessment.

12. The method of any one of the preceding claims further comprising genotyping the individual or the patient for a SERPINA1 gene mutation.

13. The method of any one of the preceding claims wherein the mutation is a PiZ mutation, optionally, wherein the patient is homozygous for PiZ mutation.

14. A method of treating or preventing AATD-LD in a subject having AATD and blood AST levels above about 34 U/L, the method comprising administering to the subject an agent that reduces AAT levels.

15. A method of treating or preventing AATD-LD in a subject having AATD and weight above about 180 pounds or below about 100 pounds, the method comprising administering to the subject an agent that reduces AAT levels.

16. A method of treating or preventing AATD-LD in a subject having AATD, blood AST levels above about 34 U/L, and weight above about 180 pounds or below about 100 pounds, the method comprising administering to the subject an agent that reduces AAT levels.

17. The method of any one of the preceding claims, wherein the agent is an siRNA.

18. The method of any one of the preceding claims, wherein the agent is TAK-999 (Fazisiran).
